(19) United States
(12) Patent Application Publication
(10) Pub. No.: US 2023/0075058 A1
(43) Pub. Date: Mar. 9, 2023

(54) WIRELESS FITNESS TRACKING INTEGRATION

(71) Applicant: North Pole Engineering Inc., Golden Valley, MN (US)

(72) Inventors: Richard D. Gibbs, Woodbury, MN (US); Tim M. Eskew, Westfield, IN (US); Richard W. Lucas, Zimmerman, MN (US); Scott B. Rodestvedt, Minnetonka, MN (US); Nicholas J. Moen, Savage, MN (US); Steffin S. Griswold, Robbinsdale, MN (US); Sebastian A. Barnowski, Cochrane (CA); Stephen E. Hidem, Edina, MN (US)

(21) Appl. No.: 17/902,232

(22) Filed: Sep. 2, 2022

Related U.S. Application Data
(60) Provisional application No. 63/240,479, filed on Sep. 3, 2021.

(51) Int. Cl. A63B 24/00; A61B 5/00

(52) U.S. Cl. CPC A63B 24/0062; A61B 5/681

(57) ABSTRACT

A mobile device associated with a user and a smart fitness equipment unit are provided, where the mobile device is a central device and the smart fitness equipment unit is a peripheral device in communication with the mobile device.

### CROSS REFERENCE TO RELATED APPLICATIONS

This application claims the benefit of and priority to U.S. Provisional Pat. Application No. 63/240,479, filed Sep. 3, 2021 the entire contents of which are incorporated herein by reference in its entirety.

### TECHNICAL FIELD

The present invention is directed to smart connectivity schemes for fitness data, environments, and tracking, which leverage smart mobile devices and connected smart fitness equipment in order to provide an integrated, seamless environment for users and devices without a need for modification of fitness equipment or other devices in order to connect mobile devices and fitness equipment to each other and/or a computer network for data display, sharing, and the like.

### BACKGROUND

At present, certain mobile devices and certain exercise equipment units can be modified or bolstered for transmission of various fitness-related data and statistics using external devices and/or sensors. However, challenges remain with respect to integration of data tracking hardware, systems, and functionality and providing seamless low-energy wireless connection options that are convenient for users. In particular, in order to establish a connection between a user’s mobile device and fitness equipment being used by the user, connections require an intermediate device that receives, converts, and rebroadcasts indirect fitness tracking data from the fitness equipment to the separate mobile device. Furthermore, existing fitness tracking setups are limited to narrow uses, both functionally and geographically.

### SUMMARY

The present invention addresses the drawbacks of the prior art by creating a seamless network of user mobile devices, such as smart watches, and providing connections between the user mobiles devices and wireless-enabled smart fitness equipment without a need for an intermediate device to facilitate communication between mobile device and fitness equipment. Furthermore, although network bridges are optional, the user mobile device can seamlessly roam over a greater geographic area through direct device-to-network bridge communication with smart hand-offs where beneficial. Yet further, the described systems and methods provide a versatile and robust framework for application to a variety of use cases and environments, such as effortless check-in or check-out or security functions at a health club or fitness facility, smart and controlled access to doors, lockers, and the like, and various notification integration, among other widespread and flexible use cases.

According to a first aspect of the present disclosure, a system is disclosed. According to the first aspect, the system includes a mobile device associated with a user. The system also includes a smart fitness equipment unit. According to the fist aspect, the mobile device is a central device and the smart fitness equipment unit is a peripheral device in communication with the mobile device.

According to a second aspect of the present disclosure, a method for providing connectivity in a fitness center environment is disclosed. According to the second aspect, the method includes providing a plurality of mobile devices. The method also includes associating the plurality of mobile devices with a corresponding plurality of users. The method also includes providing a plurality of smart fitness equipment units. The method also includes creating a network connecting at least one of the plurality of mobile devices and at least one of the plurality of fitness equipment units. Also, according to the second aspect, each mobile device of the plurality of mobile devices is a central device and each smart fitness equipment unit is a peripheral device in direct communication with at least one central device of the plurality of mobile devices.

According to a third aspect of the present disclosure, a method of connecting a user mobile device to a smart fitness equipment unit is disclosed. According to the third aspect, the method includes broadcasting an availability signal from a peripheral smart fitness equipment unit. The method also includes listening for an availability signal using a central mobile device. The method also includes initiating a handshake operation between the peripheral smart fitness equipment unit and the central mobile device. The method also includes establishing a direct bi-directional connection between the peripheral smart fitness equipment unit and the central mobile device.

### BRIEF DESCRIPTION OF THE DRAWINGS

FIG. 1 is a schematic view of an existing scheme that utilizes a sensor pod for intermediate connection between a user mobile device and fitness equipment.

FIG. 2 is an example of a connected fitness system, according to various embodiments.

FIG. 3 is a schematic view of a fitness environment connectivity and network scheme, according to various embodiments.

FIG. 4 is a schematic view of another fitness environment connectivity and network scheme, according to various embodiments.

FIG. 5 is a schematic view of a data cloud to network bridge interface of a fitness environment connectivity and network scheme, according to various embodiments.

FIG. 6 is a multi-level view of various connections and devices for use in a fitness environment connectivity and network scheme, according to various embodiments.

FIG. 7 is an overview of a fitness center environment with a network scheme integrated therewith, according to various embodiments.

FIG. 8 is a view of a fitness machine console showing a connection to a user mobile device, according to various embodiments.

FIG. 9 is a view of a fitness machine console showing a mirrored display and linked connection to a user mobile device, according to various embodiments.

FIG. 10 is a view of a fitness machine console showing a connection interface display for a user mobile device, according to various embodiments.

FIG. 11 shows various views of a user mobile device according to various stages of use, according to various embodiments.

FIG. 12 shows example metrics communicated by or with a fitness equipment console, according to various embodiments.

FIG. 13 is a top view of a network bridge printed circuit board, according to various embodiments.

FIG. 14 is a top view of a wireless connectivity module printed circuit board, according to various embodiments.

FIG. 15 is a front view of a wall plate for installing a network bridge, according to various embodiments.

FIG. 16 is a rear view of the wall plate of FIG. 15, according to various embodiments.

FIG. 17 is an example wiring pinout chart for a network bridge, according to various embodiments.

FIG. 18 shows an example preferred position for a network bridge in a room of a first size, according to various embodiments.

FIG. 19 shows an example preferred position for two network bridges in a room of a second size, according to various embodiments.

FIG. 20 is a fitness environment connectivity and network scheme process, according to various embodiments.

### DETAILED DESCRIPTION

Disclosed are embodiments of fitness tracking and data sharing and distribution methodologies, systems, and environments that provide seamless integration with various connected devices, utilizing elements of existing wireless technologies and standards, and that is preferably bolstered by back-end networking for cloud and/or Internet connections.

Proliferation of mobile devices, including wearable devices and smart watches, continues. Mobile devices, especially smart watches, often now include a variety of biometric data sensing and logging capabilities, such as heart rate, pedometer, accelerometer, electrocardiogram (EKG), altimeter, among others. Other, non-smart-watch, mobile devices, such as mobile phones, bike or other equipment computers, and pedometers, can provide at least some degree of user biometric data gathering. Therefore, it would be beneficial to leverage these existing and versatile mobile devices for use in various environments where user biometric data is of interest or use. One such example environment exists in or is associated with a health center or fitness club setting or facility. Although many embodiments herein are directed to use in fitness club facilities in particular, it is contemplated that a user could benefit from methods and systems herein in any number of other settings, such as at home, at school, at a library, a shopping mall, in an urban environment or park, at an office or work site, a sports arena or field, or any other location or facility type as appropriate.

Embodiments of the present disclosure build upon wireless architectures, in particular Bluetooth Low Energy (BLE), and introduce a new wireless connection methodology where a user mobile device is configured to be a BLE “central” or primary device to which various BLE “peripheral” or secondary devices are connected. By selecting device roles such that a user’s mobile device, such as a wearable or smart watch device, becomes the BLE central device, existing frameworks and permissions are usable to bolster biometric sensing and tracking data from the central mobile device for wireless distribution and connection to various other wireless-equipped BLE peripheral devices, including wireless communication devices integrated into or otherwise interfaced with fitness equipment and/or consoles thereof.

In more detail, a central device, as defined by the BLE standard, is a wirelessly-enabled device that discovers and listens to other devices that are “advertising” (i.e., seeking connections by sending [e.g., TCP/IP] data packets containing data for other devices to receive and process). The central device can be configured to connect to one or more advertising peripheral wirelessly-enabled devices. A peripheral device for BLE is a device that advertises for connections and that can accept connections from central devices. According to the BLE standard, a total of 40 channels are or can be used for communication, of which three channels are used for advertising as “primary” advertising channels.

BLE is a standard managed by the Bluetooth (BT) Special Interest Group (SIG). The SIG maintains a library of standardized and published services and characteristics, which are intended to promote a common use case amongst developers and applications. These standards also exist to promote interoperability. Because of the standards set by the SIG, developers are typically locked into a standard method of implementation in which the “peripheral” device is the generator of measurement data and the “central” device collects the data.

Some terms used herein are defined as follows:

ANT: ANT is a proven ultra-low power (ULP) wireless protocol that is responsible for sending information wirelessly from one device to another device, in a robust and flexible manner.

ANT+: ANT+ is a set of mutually agreed upon definitions for what the information sent over ANT represents.

Apple Watch: An Apple-branded smart watch mobile device that runs Apple Inc.'s watchOS (based on iOS), and is equipped with Bluetooth technology, including Bluetooth Low Energy (BLE). Due to hardware limitations, some embodiments of the smart connectivity ecosystems described herein preferably work best with Apple Watch Series 2 and newer.

BLE: Bluetooth Low Energy is a low-energy wireless connectivity standard that can operate in parallel with (higher-energy and higher-bandwidth) Bluetooth Standard, both equipped in most mobile devices since 2010.

BLE Central: Bluetooth Low Energy radio implementation setup to listen for an advertisement from a BLE Peripheral device. Some devices, such as the Apple Watch are configured to operate only as a BLE Central device, and not a BLE Peripheral device.

BLE Peripheral: BLE radio implementation setup to broadcast an advertisement announcing itself to allow a BLE Central device to request and establish active bi-directional communication by initiating a handshake operation between the peripheral and central devices. The Apple Watch is not configured to operate as a BLE Peripheral device.

Heartbeatz: A proprietary BLE smart connectivity framework and network ecosystem developed by Applicant (North Pole Engineering, Inc.) that is configured to connect BLE Central only devices to various biometric or heart rate collection implementations. The Heartbeatz trade name and the more general term “smart connectivity ecosystem” are used interchangeably herein.

Smart Watch: A typically wrist-worn wearable mobile device equipped with BLE capable of connecting to a smart phone or other wireless enabled device.

Samsung Gear: A Samsung brand smart watch mobile device that supports fitness operation and features.

WASP-N: Applicant’s proprietary Wi-Fi-based ANT and/or BLE data transceiver, also referred to herein as a network bridge.

WASP-PoE: Applicant’s Power Over Ethernet (PoE)-based ANT and/or BLE data transceiver, also referred to herein as a network bridge or a PoE network bridge, herein.

WASP3: Applicant’s Power Over Ethernet (PoE) based ANT and/or BLE data transceiver, also referred to herein as a network bridge or a network bridge (version 3) herein.

GEM(1,2,3): Applicant’s wireless communications and connectivity modules, with the appended number indicating a version or generation number of each GEM. The various GEM module versions refer to a proprietary wireless connectivity module developed by Applicant, and “GEM” and “wireless connectivity module” can be used interchangeably herein. A GEM module can be used with Applicant’s smart connectivity ecosystem mobile device application to send heart rate and calories data to a GEM2 enabled fitness equipment console for display and for the GEM2 to send workout metrics and equipment control signals from the console to the smart connectivity ecosystem mobile device application for workout logging and application start/stop/pause control. The wireless connectivity module(s) can be installed in a network bridge (e.g., a WASP network bridge), various fitness equipment, front desk or other computer-enabled settings. The firmware in a wireless connectivity module (version 3) can be utilized in the network bridge (version 3) and supports 15 concurrent connections of the smart connectivity ecosystem interface. A network bridge 118 (version 3) therefore, if equipped with four wireless connectivity modules, can simultaneously connect to 60 (15 per module) separate mobile devices. Additional modules can be incorporated into example network bridges, and more connections per module are also contemplated.

GEM3-PoE: Applicant’s Power Over Ethernet (PoE) based ANT and/or BLE data transceiver, also referred to herein as a wireless connectivity module (version 3) herein.

Described herein are embodiments that utilize wireless devices and network systems and protocols beneficially and counterintuitively. Disclosed protocols and schemes counterintuitively reverse the standard paradigm, and allow the BLE central device to instead be the originator of the data of interest, such as biometrics data. As a significant breakthrough, embodiments of the present disclosure provide a mechanism for utilizing existing and familiar user devices seamlessly for fitness, identification, and access control use without a need for a wireless conversion, e.g., from BLE to ANT+ wireless standards for retransmission. However, it is noted that a user need not use an existing device of the user in all cases. For example, a health club facility or other environment can provide a mobile device capable of tracking biometrics to a user when a membership contract is signed or the like.

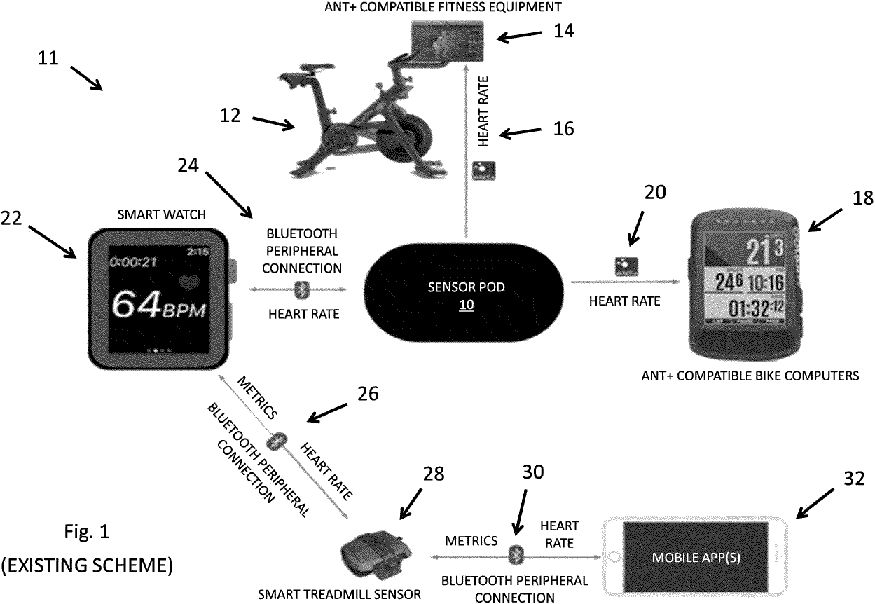

One existing connectivity scheme is shown at scheme 11 of FIG. 1. As shown in the illustrated existing scheme, a user’s smart watch 22 is connected to a fitness machine 12 with console 14 and an ANT+ wireless compatible bike computer 18 via an intermediate sensor pod 10. As shown, the smart watch 22 connects to the pod 10 via a Bluetooth peripheral connection 24, the fitness machine 12 and console connect to the pod 10 via an ANT+ connection 16, and the bike computer 18 connects to the pod 10 via an ANT+ connection 20. In the shown existing scheme, a smart treadmill sensor 28 (e.g., a Runn product sold by Applicant, North Pole Engineering, Inc.) is installed on a treadmill (not shown) for wireless communication with the smart watch 22 via Bluetooth wireless peripheral connection 26. The smart treadmill sensor 28 is then connectable to a smart phone 32, including mobile application connections, via Bluetooth peripheral connection 30.

Various examples of currently-available hardware are commercially available in the following example products offered by Applicant: Heartbeatz Pod, CABLE, OTLink, and Runn. Various existing products utilize a separate communications unit in combination with a user mobile device to achieve full connectivity. Specifically, each of these products operates as the bridge to make the smart watch heart rate data available to external ANT+ receivers. For example, Applicant’s Runn product is a mountable product that could be attached to, e.g., a side of a treadmill with a cradle interface. Additional sensors could be attached to the treadmill in order to adapt the treadmill for smart use. The resulting system can provide real-time metrics from a user’s workout, such as speed, pace, distance, incline, cadence, heart rate, etc.

Applicant’s existing smart connectivity ecosystem and interface (which is adaptable for use in present embodiments) connects a user’s sensed heart rate data from a mobile device such as a smart watch and provides a wireless connection (e.g., via ANT+, BLE, etc.) to various exercise equipment interfaces, such as a fitness machine console. The existing smart connectivity ecosystem converts biometric data sensed at the mobile device and converts the data to a real-time wireless data stream to be received by another networked device. A user can source and install the smart connectivity ecosystem connectivity to a mobile device such as a smart watch using a second mobile device such as a mobile phone or tablet device. The second mobile device can provide a more robust display for analysis or data display associated with user data.

Applicant’s smart connectivity ecosystem mobile device application technology enables two-way Bluetooth communications between the smart connectivity ecosystem mobile device application and devices such as Applicant’s smart connectivity ecosystem ANT+ HRM sensor pod, a smart treadmill sensor, wireless connectivity Bluetooth and ANT+ OEM module, and an ANT+ and Bluetooth sensor network bridge. With this two-way (bi-directional) direct communication link, real-time heart rate and calorie data measured by the mobile device can be shared with the smart connectivity ecosystem compatible device. In addition, data, messaging, and control signals can be sent from the smart connectivity ecosystem compatible device to the smart connectivity ecosystem mobile device application for logging and enabling application messaging and control functions within the smart connectivity ecosystem mobile device application.

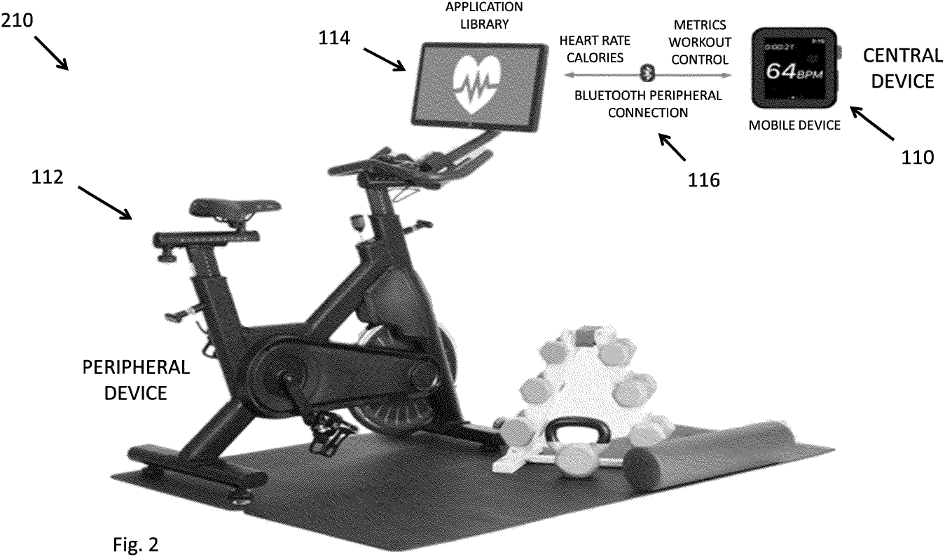

Turning now to embodiments of the present invention, FIG. 2 shows a simplified view of an example scheme 210 that utilizes the connection methodologies of the present disclosure. As shown, a user mobile device (e.g., a smart watch or other wearable device) 110 is configured as a BLE central device, and is connected directly to a BLE peripheral fitness equipment machine (or unit) 112 and console 114 via a BLE connection 116 and as shown without the use of a network bridge (118 or the like). The (peripheral) fitness equipment machine 112 is optionally an Android-OS-based consumer fitness equipment unit, such as a stationary bike. The console 114 preferably includes a hardware processor operative connected to a memory, which is also preferably loaded with an application library, such as an Android-OS-based library corresponding to the fitness equipment machine 112, as shown.

The BLE connection 116 can transmit various metric data, including heart rate, calories, and various workout control aspects, among other data. Data transfer between the central mobile device 110 and the peripheral equipment 112 is preferably capable of direct bi-directional communication once connection is initiated. In this way, the need for a network bridge as shown is eliminated, as the functionality of a network bridge is incorporated into the console 114 to allow the mobile device 110 via BLE connection 116 to act as a central device (instead of a peripheral device as is currently typical) and to directly communicate with the console 114 of the equipment 112 (which acts as a peripheral device instead of a central device as is currently typical). As can be seen, scheme 210 is greatly simplified when compared to the existing scheme including an intermediate pod 10 as shown in FIG. 1.

A key detail of the connectivity technology described herein is the unique and new implementation and use of the existing BLE infrastructure. The typical and existing use case for adding a monitoring device (e.g., heart rate monitor [HRM]) to a system has the HRM operating as a BLE “peripheral” device with the HRM BLE service used as the transport mechanism to a central device that can communicate with the HRM. Present embodiments therefore counterintuitively reverse the existing methodology, and flip the roles of the HRM and device receiving the HRM (or other biometric) data. In other words, as described herein, the HRM operates as the BLE central device and the system consuming the data is defined as the BLE peripheral device (e.g., a fitness equipment unit or machine 112 or console 114 thereof). This has a significant benefit of reducing or even eliminating the use of network bridges 118 as described further herein.

This breakthrough use of the BLE connection allows a commonly-available and widely adopted consumer mobile device 110 (e.g., a smart watch) which does or may not support being a BLE peripheral device to supply heart rate or other biometric or user data outside its proprietary platform. This is a major improvement in that it allows a user to use existing hardware and devices in a completely new and seamless way.

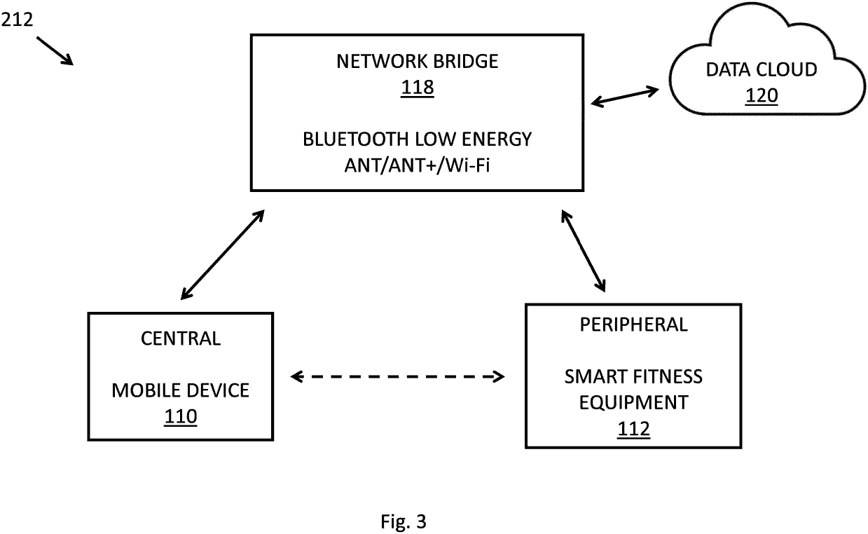

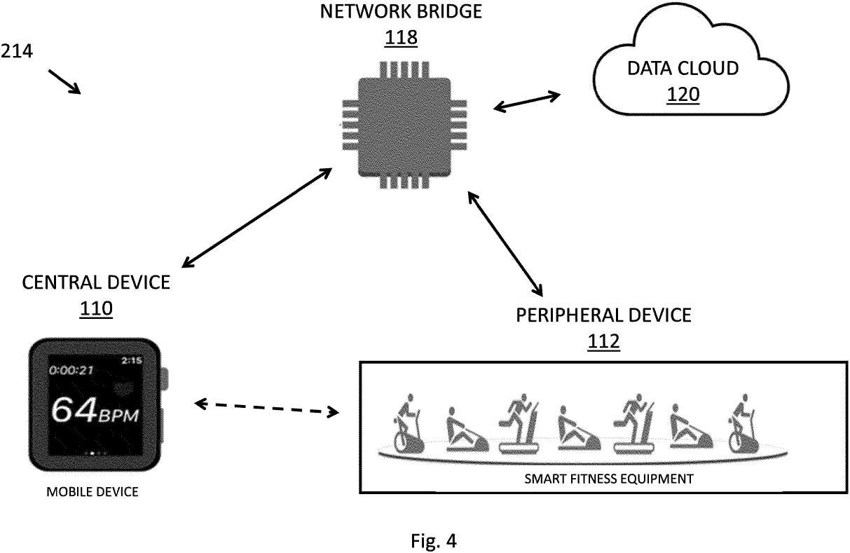

As shown in fitness environment connectivity and network schemes 212 and 214 of FIGS. 3 and 4, disclosed connectivity technology can be used to interface a mobile device 110 (e.g., a smart watch) capable of measuring a user’s heart rate with other peripheral devices 112 via seamless wireless connections. The disclosed connectivity technology can further include specific purpose-built products and devices configured to relay user biometric data (e.g., HRM data, blood pressure, steps, etc.) to other connected devices, such as optional wireless network bridge devices 118 capable of receiving user data wirelessly (e.g., via ANT+, BLE, Wi-Fi radio protocols).

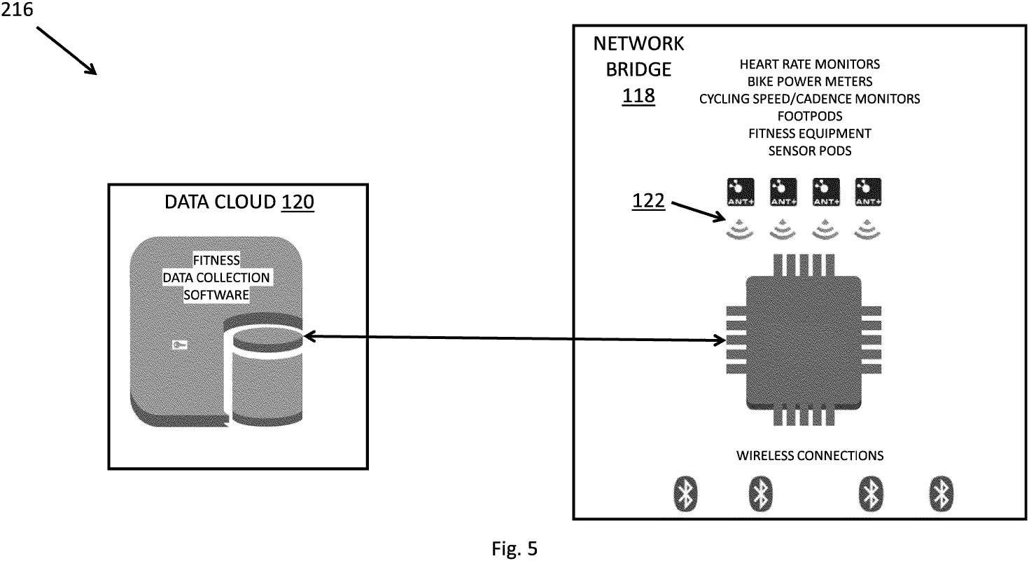

If present, any network bridges 118 can also preferably in wired and/or wireless network connection with a back-end data cloud 120 via one or more wireless connectivity modules 122 as shown in scheme 216 of FIG. 5 (four modules 122 as shown). The data cloud 120 can include one or more computers equipped with one or more hardware processors operatively connected to one or more memories, storage devices, displays, input devices, network communication modules, and the like.

Various defined device types and roles are discussed herein, include a 1. User mobile device (central, mobile device 110), 2. Wireless-enabled fitness equipment or machine (peripheral device 112), 3. Network bridge 118, 4. Wireless module 122 (e.g., GEM, as described herein), and 5. Back-end server/data cloud 120. Each is discussed briefly in turn, below:

User (central) mobile device 110: a user mobile device is associated with a user, and preferably includes a wearable mobile device such as a smart watch. In various embodiments, a user mobile device 110 can be a purpose-built biometric monitor, such as an HRM or the like, and the user mobile device 110 can be a smartphone in other embodiments. Preferably, a user provides the mobile device 110 for wireless access and connection to other wireless-enabled mobile devices via a wireless interface. Example mobile devices 110 include smart watches, such as Apple Watch, Samsung Gear, mobile phones, pedometers, other wearable devices, among many others.

Wireless-enabled fitness equipment, machine, or unit 112 (such as a peripheral device as described herein) can be any of various exercise machines, equipment, and units (such terms are used interchangeably, herein), each of which are contemplated herein. Some examples include treadmills, stationary bicycles, rowing machines, elliptical machines, among many others. Either from factory or by retrofit, the fitness equipment 112 can include one or more wireless connectivity module 122 as described further herein. The fitness equipment 112 can include a console 114 portion with a processor, memory, user interface and display, and interface for connection to the wireless connectivity module or components.

One or more example network bridges 118, such as the “WASP” units manufactured and sold by Applicant, can optionally be deployed in association with various buildings or facilities. Various infrastructure data connections, such as data collection endpoints, wireless network bridges, or wireless access points, are used in present embodiments. One such wireless network bridge 118 contemplated herein incorporates ANT+ and BLE into a smart connectivity ecosystem compatible network sensor bridge, and such data collection device is capable of connecting to multiple mobile devices 110 of multiples users, concurrently and simultaneously.

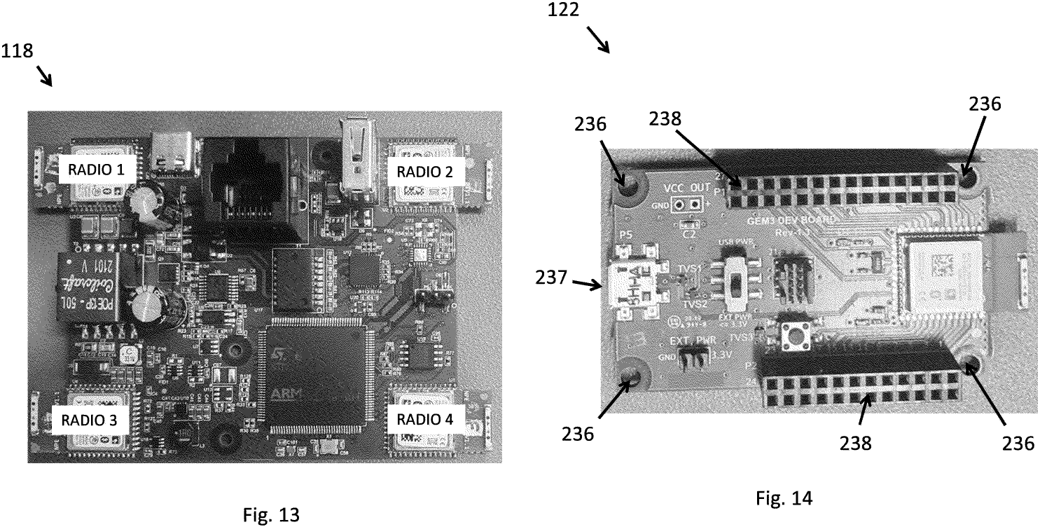

The network bridges 118 (if utilized) are preferably equipped with one or more, e.g., four, wireless connectivity modules 122 for wireless access and transmission control. For example, in FIG. 13, a network bridge is shown with four radios 1-4, which each can be an example of a wireless connectivity module 122 as used herein. The network bridges 118 (if present) can be interconnected using, e.g., power over ethernet (PoE), wirelessly, through conventional wired networking and power connections, or any combination thereof. The network bridges 118 are configured to communicate with one or more user mobile device 110, one or more fitness equipment machines 112, and optionally with a back-end server or cloud 120, through wired and/or wireless network connections.

A wireless connectivity module 122, (such as various versions of the GEM units manufactured and sold by Applicant), is preferably a self-contained wireless module and microchip configured to provide wireless communication and networking connections and connectivity to a network bridge 118, fitness equipment 112, or another device (e.g., mobile device 110) that can utilize wireless communications, e.g., by BLE or ANT+, according to various embodiments herein.

A back-end server, system, or cloud 120 is contemplated herein. For cloud 120, any number of servers, server farms, or the like can be utilized according to need and usage parameters. The back-end server can provide Internet-based access to user data tracking statistics and analysis, and can provide secure log-ins for users, health clubs, or other authorized users to view and/or manipulate user fitness tracking data. In some examples the cloud 120 is built on the Heartbeatz software developer kit (SDK) ecosystem developed by Applicant.

For embodiments herein, a user mobile device 110 can be referred to as the “central” mobile device 110 to which a “peripheral” device 112 is connectable. Examples of peripheral devices 112 contemplated herein include connected exercise or fitness equipment or consoles thereof associated with smart fitness equipment 112, such as treadmills, stationary bikes, rowing machines, and the like.

Example wireless connectivity devices, such as modules 122, can be used to bridge the user data from the mobile device 110 to another smart device, such as smart fitness equipment 112, units, or systems, preferably operate using the BLE standard (or similar). BLE-equipped “peripheral” devices allow the central mobile device 110 to connect to them using their BLE central radio and send the heart rate data to them for rebroadcast to the network bridge BLE/ANT+ receiver.

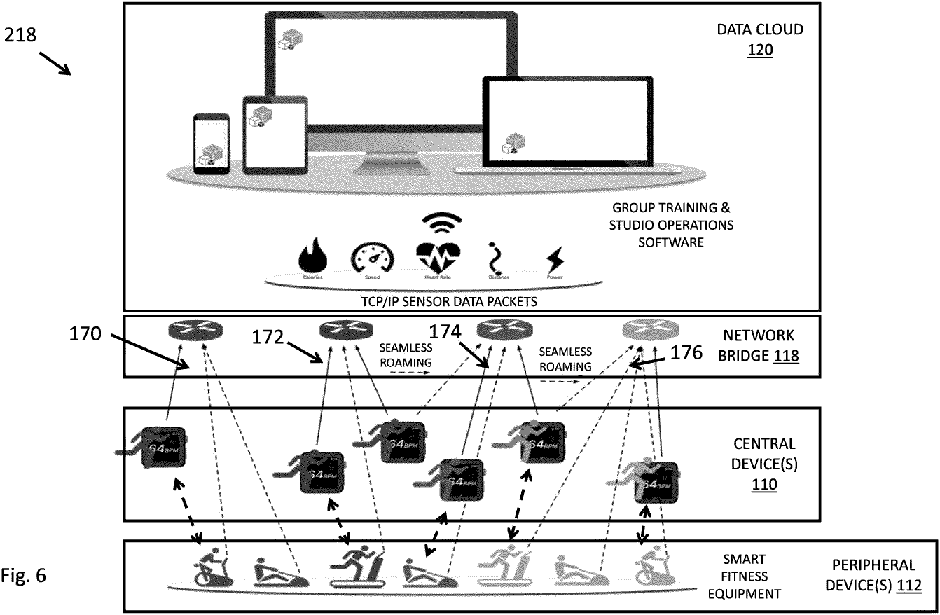

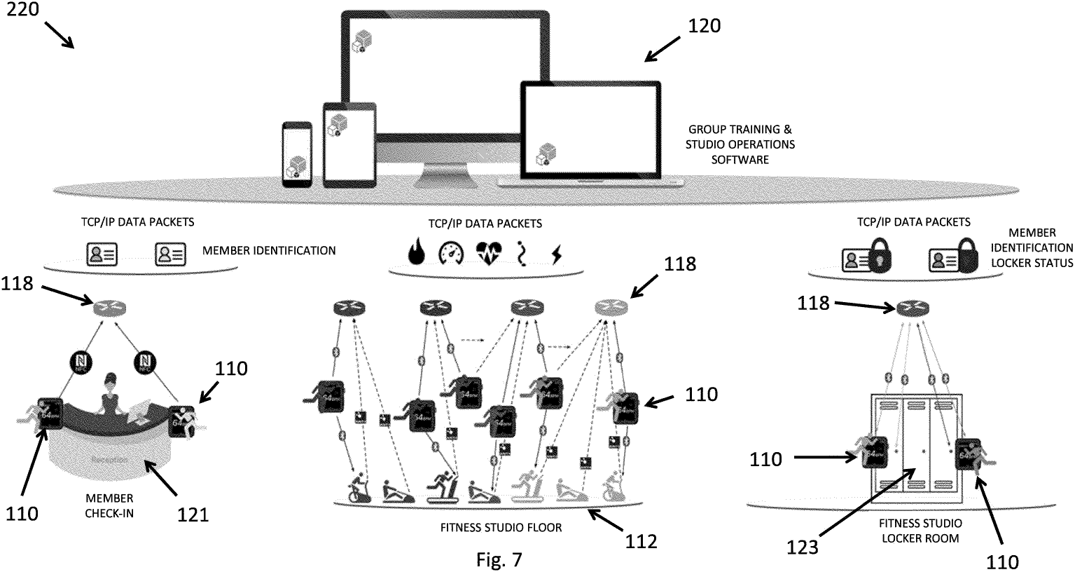

With reference to FIG. 6, a high-level view of a wireless-enabled (fitness) environment is shown at 218. A broader view of a related wireless-enabled environment is shown at 220 of FIG. 7. As shown, embodiments of the present disclosure provide significant improvements to existing connectivity technology by simplifying and making connections more direct and more seamless. Embodiments described herein allow the elimination of need for an additional consumer product to allow the flow of data from the mobile device 110 operating as a BLE central device to the endpoint collector of the of the heart rate data running as a BLE peripheral device 112. Disclosed methods and systems allow the mobile device 110 associated with a user to connect directly to an existing properly equipped data collection endpoint (wireless network bridge 118, if present) or directly to a device equipped with a BLE radio setup as a BLE peripheral device (smart fitness equipment 112).

As an overview of an example smart connectivity ecosystem, the wireless connectivity module (version 2), when integrated into a fitness equipment device, can pair with a compatible mobile device using a “Bluetooth proximity” method where by the mobile device is brought near an area of the fitness equipment console 114 where the wireless connectivity module 122 has been located. The mobile device 110 running Applicant’s smart connectivity ecosystem module will sense the presence of the wireless connectivity module and open a Bluetooth connection automatically. Once the connection has been established between the smart connectivity ecosystem application on the mobile device 110 and the wireless connectivity module 122, the console 114 and mobile device 110 can exchange workout data using the Bluetooth smart connectivity ecosystem service.

In one example, to establish a wireless (e.g., Bluetooth) connection, the smart connectivity ecosystem technology can utilize proximity features/sensing to ensure that the mobile device 110 owner connects their mobile device 110 to the desired wireless connectivity module enabled device. The smart connectivity ecosystem mobile device application scans for the smart connectivity ecosystem advertisement messages being sent by the wireless connectivity module 122 when Bluetooth advertising is initiated/enabled by the fitness equipment console. The smart connectivity ecosystem application will automatically connect to the wireless connectivity module when the received signal strength indicator (RSSI) of the Bluetooth advertisements is high enough to trigger the smart connectivity ecosystem application to initiate the Bluetooth connection. The connection range in the smart connectivity ecosystem application is approximately 1-2 inches. Once the initial Bluetooth connection has been established, the mobile device owner can move their mobile device 110 away from the fitness equipment console 114 and exercise at a normal distance away from the fitness equipment console 114 without risking that the wireless connectivity module 122/mobile device 110 Bluetooth link will be disrupted.

Since the optional wireless network bridge 118 or endpoint can connect to multiple devices 110/112 concurrently, data can be routed bi-directionally between the device(s) to the wireless network bridge 118 collection system and between the individual mobile device(s) 110 connected to the system. In other embodiments, no network bridges 118 are utilized. Additionally, embodiments are contemplated with multiple collection endpoints in the form of wireless network bridges 118 in the overall system 218 shown in FIG. 6. In such environments, and broader environments such as a health club or fitness center facility shown in FIG. 7, the user’s mobile device 110 can beneficially and automatically roam seamlessly, including a hand-off and switch from the currently connected wireless network bridge 118 endpoint to another wireless network bridge 118 endpoint if and when the device determines there is a stronger single path between the device and a different data collection endpoint.

In other words, the mobile device 110 can selectively determine that a better wireless network bridge connection is or is not available and selectively switch on its own as it deems beneficial. This hand-off of central mobile device 110 connections (and optionally peripheral device 112 connections) between more than one network bridge 118 can be referred to as roaming and is shown in FIG. 6 as a user and associate central device move between various fitness equipment 112 and associated zones 170, 172, 174, and 176 associated with each respective network bridge 118. When the network bridge 118 connection switches for the user’s mobile device 110, data communication is automatically routed from the new wireless network bridge 118 endpoint to the main processing endpoint in the system, e.g., the cloud 120. This self-contained switching determination is in contrast to, e.g., typical cellular device connections where tower-tower hand-offs are at least partially influenced by cellular tower intercommunication and preferences.

As shown in FIG. 6, a user represented by an icon is shown in process of transitioning or “roam” from one wireless network bridge 118 or data collection node to another seamlessly, and transitioning from zones 170, 172, 174, and 176. Although various wireless network bridges 118 can be positioned near various exercise equipment 112 locations, a user can in some cases transition from wireless network bridges 118 while using a machine 112 or in some cases transition to a new machine 112 while maintaining a connection to the same wireless network bridge 118.

In an example, a user has a mobile device 110 that is already connected to a network bridge 118 in a (e.g., fitness) facility for communication with various other devices of the facility. In this example, the user may desire to interact directly with a peripheral smart fitness equipment device 112. The user can concurrently connect the central mobile device 110 directly to the peripheral device 112 (e.g., treadmill) to allow the user heart rate and other biometric tracking data, such as calorie burn and cadence, to be sent to the peripheral device 112 (treadmill) for display on the console 114, and processing at the peripheral device 112, and further to allow the central mobile device 110 to receive peripheral device 112 data such as speed, incline, and distance (e.g., for a smart treadmill) to be recorded in the workout being tracked by the user’s central mobile device 110. Any relevant peripheral device 112-generated data can then also be sent from the watch to the network bridge 118, e.g., for consumption by a facility workout recording/display system with the data publicly or privately associated with the specific user’s account or profile. In other words, the central mobile device 110 can communicate with various peripheral devices 112 directly or indirectly in various embodiments and situations.

In various embodiments, multiple central mobile devices 110 can communicate with each other when the mobile devices 110 are connected to another device (e.g., a network bridge 118, peripheral device 112, etc.) capable of either having multiple mobile devices 110 connected (e.g., 15 devices) to the devices' BLE peripheral connections, or when the mobile device 110 is connected to the peripheral connection of a device 112 connected to an optional network bridge 118 infrastructure. The connection can be formed where the devices providing the peripheral connection (e.g., peripheral devices 112) have communication capability with the other peripheral devices over the network formed by the network bridges 118 and other features described herein.

Embodiments of the present disclosure therefore offer improvements over the existing schemes. In the case of the existing devices that, e.g., utilize an intermediate device (such as the sensor pod 10 described in FIG. 1 and the like), the mobile devices 110 may not have a communication path to interact with other similar mobile devices 110. Mobile device 110 to mobile device 110 communication can, however, be established in some cases using platform-proprietary technology, such as “multi-peer connectivity” (in the case of the Apple’s iOS environment), and in that case, the mobile device 110 can communicate through a second mobile device of the same user (e.g., a phone paired to an associated smart watch) as a bridging device for retransmission.

With reference again to FIG. 6, shown are various examples of connected fitness equipment (peripheral devices) 112 sending data to optional individual wireless network bridge 118 data collection nodes using (e.g., ANT+ or BLE) with users having mobile devices 110 associated with each user, and each device using a contemplated wireless protocol or smart connectivity ecosystem. As described herein, a user’s mobile device 110 preferably operates as a central BLE mobile device and forms a paired connection with relevant peripheral exercise equipment 112 concurrently with the mobile device 110 and machine 112 communicating with the appropriate wireless network bridge 118 node.

Mobile devices 110 and equipment 112 of different ecosystems can also beneficially intercommunicate, according to various embodiments. In the case of the direct connection to a display device using a BLE radio as a peripheral device, the disclosed connection technology allows mobile devices to seamlessly transition from different ecosystems to interoperate with one another. For example, using presently disclosed connectivity technology and a user-wearable mobile device 110 (e.g., a smart watch, such as an Apple Watch), a hardware device equipped with an enabled application can send heart rate and other data information directly to another mobile device 110 (e.g., running Android operating system) using a loaded (e.g., Android) library. In another example, a mobile device 110, such as a Samsung Gear brand smart watch can form a bi-direction connection with another mobile device 110. For example, the first mobile device 110 can connect to and send user data or biometric data (e.g., user heart rate information) to another mobile device 110, such as an Apple iPhone running the appropriate application via the iOS library.

The bi-directional connection can be used to have the first mobile device 110 control the operation of a mobile application, or the mobile application can control the operation of the second device’s mobile device application. In some embodiments, one central mobile device 110 of a first user can control applications on another device, such as another device of the same user or another device associated with a different user, However, any such communication is preferably authenticated using a secure communication protocol before being allowed to send control commands to the connected application. Any other wearable, mobile, or smart mobile device 110 can be interfaced with the optional wireless network bridges 118, and/or any other device or system described herein.

A broader example showing how the disclosed connectivity technology can be used throughout a fitness center, environment, or facility, is shown in diagram 220 of FIG. 7. In this example, a user has a mobile device 110 equipped with a properly enabled application that is configured to connect to the optional network bridge 118 or other data access point as a check-in function, e.g., at a front reception area 121, e.g., at a main entrance. The check-in location can be associated with another network bridge 118, and in some optional embodiments various near-field communication features can be incorporated into this or any other steps described herein. As the user’s mobile device 110 gets closer to the front desk 121, the mobile device 110 can automatically connect to the network bridge 118 node there and can identify itself (and by association, the user) to the facility’s check-in system.

An example application for proximity or a near-field communication (NFC) enabled module 122 within a fitness facility is at the front desk for checking in members. A reception or check-in situation is one example of a “proximity mode” that can perform other NFC-type functions without a need for cumbersome, very close proximity between devices. The NFC version of the module 122 can read user membership cards and transmit this information to the back-end system (cloud 120) over a wireless network formed by network bridges 118 or through a wired direct connection using, e.g., a USB connection to the back-end (cloud 120 or server) software system.

In some embodiments, a reception 121 station can be equipped with various (e.g., NFC-enabled) hardware that allows for proximity of the user mobile device 110 to cause the user’s information to be automatically displayed onto a reception 121 terminal allowing front desk staff (or computerized access control) to validate the user as a client, e.g., based on user photo, name, etc., and greet/acknowledge the user by name without the need to directly scan the user’s membership card using a barcode, with direct device contact, or otherwise slow down the validation and welcome process. The user mobile device 110 equipped with the described smart connectivity ecosystem and network connectivity can also be used to securely interact with individual locks and other features that can be access-controlled.

Another application for a contemplated system with a network bridge 118/module 122 combination in a facility is locker 123 or storage access control. Modules 122 could be integrated into locker 123 lock assemblies and the mobile device application could be used to selectively open/close/unlock/lock a locker 123, provided access is granted by the system. The network bridge 118 could monitor the lock status, access rules, and report the data to a back-end system (cloud 120) that preferably implements a software developer kit (SDK), such as Applicant’s WASP SDK. Yet another example is using the systems described herein for purchase, e.g., using smart vending machines or smart ordering and the like.

Therefore, access control on a micro level within a fitness club can extend to private lockers 123 as shown in FIG. 7. In other embodiments, a lock to a door that would provide access to secure locations can also utilize the BLE communication via the user’s mobile device 110. Using the smart connectivity ecosystem link in a proximity mode, the mobile device (e.g., smart watch) can be held close to the locker 123 or access panel and use the secure connection to unlock the locker 123 or gain authorization to access a space, such as by a doorway to a part of a facility only available to “premium” members, parents of certain children, or other select members. In another example, a premium “club” area may have an access door only unlocked for premium members with their mobile device 110 indicating access is granted. In yet another example, after-hours access can be granted to various members, staff, emergency personnel, etc. according to various access guidelines and rules.

The properly-equipped (central) mobile device 110 can also be used to perform automatic check-in to a class or activity within or associated with a facility. When the mobile device 110 gets into proximity of the space identified for the activity, the mobile device 110 can be configured to automatically connect to the check-in network bridge node and cause a signal to announce itself to the system. The user can then be notified via the mobile device 110 of the successful check-in via the mobile device user interface, which can allow the user to accept or reject the operation. It is noted that any proximity or other smart function of the properly equipped mobile device 110 can optionally utilize user-validation, such as with a yes/no prompt, as appropriate in various cases.

Challenges have also existed with respect to device pairing and/or authentication. A very common and complicated process in all group fitness applications involves adding a user’s personal mobile device 110 to the system so the user’s data can be properly and securely attributed to their account. According to the present disclosure, the user’s (central) mobile device 110 can interact with the facility system via optional network bridge(s) 118 to allow for positive feedback and confirmation of a successful pairing without the need for additional equipment beyond the installed facility infrastructure, such as the network bridge 118 and back-end servers of cloud 120. In various embodiments and as shown in FIGS. 6 and 7, various computers and computer-enabled devices can be comprised within cloud 120, and optionally one or more devices thereof can be loaded with various SDKs and the like.

An example process begins with the user activating a smart fitness and systems enabled application on their mobile device. The device then finds a peripheral equipment 112 (e.g., a smart connectivity ecosystem peripheral endpoint), such as described herein, and connects to the network bridge 118 node. At this point, the central mobile device 110 of the user can send a unique, e.g., 32-bit identifier to the network bridge 118 and back-end system (e.g., cloud 120). The back-end system can then identify the connecting central mobile device 110 as a new ID and can initiate a pairing process. Once the central mobile device 110 has been allowed to join the facility network via the network bridge 118, the mobile device 110 can sends a user-specific ASCII string to the system to aid in the identification process, e.g., of the user associated with the mobile device 110. When the user recognizes their user-specified ASCII string, such as a first name or username, the facility system can selectively validate the user and optionally can send its own unique 32-bit ID code to replace the code generated by the user’s mobile device 110. If there is more than one user validating at the same time and they happen to have the same ASCII (alphanumeric) user identifier string, such as “John,” the system can send a message, sound, or haptic alert to an individual user’s mobile device 110 to allow them to uniquely identify themselves, e.g., as “John S.,” or “John-2.” This bi-directional positive feedback approach is unique to the mobile device 110 identification process and is enabled by the bi-directional nature of smart connectivity implementation and schemes as described herein.

Group settings and group events such as fitness classes or sports competitions are also bolstered using various embodiments of the present disclosure. For an example group fitness class or session, the various users can each use his or her mobile device 110 to communicate with the group data collection system via the network bridge 118 without the need for each user to bring or activate a smart phone or another secondary bridging device. Group fitness classes can involve an instructor-led session and can optionally include a leaderboard displayed on a wall or screen that shows various metrics individualized or aggregated anonymously from the class participants. Likewise, a sporting event or competition can produce athlete data.

A group fitness session can also optionally be distributed using a network such as the internet to connect the users together in a real-time fashion. In some embodiments, a user’s mobile device 110 is configured to measure biometrics of the user and the mobile device 110 can be interfaced to a wide area network (WAN, e.g., the Internet) in a separate location to send data to a session operating in a facility allowing remote users to send signals and/or remotely participate in a group fitness class that can simulate the user being present in the facility.

Embodiments of the present disclosure employ flexible, cross-platform support. Various platforms used on mobile devices include Apple watchOS/iOS, Alphabet’s Android OS, and various other Linux, Windows, and other operating systems. A user equipped with a mobile device, such as a smart watch, can connect and interface directly or indirectly with another user equipped with a mobile device such as an Android OS based device. Likewise, a user with an Android OS-based mobile device can optionally connect to a watchOS/iOS-based mobile device for any purpose, such as sending workout metrics bi-directionally between the two disparate eco-systems without the need for any additional bridging hardware. Although Android-based embodiments are shown and described herein, it is to be understood that any other OS, API, language, or systems may be used with disclosed embodiments.

A user mobile device 110 can interface with and connect to a fitness equipment console 114 of a fitness machine 112. For example, a user with a smart watch mobile device 110 can connect to a fitness equipment console 114, e.g., of a treadmill fitness unit 112, without a requirement of any eco-system approval agreements in various embodiments. This flexible connection model allows equipment manufactures to seamlessly and effortlessly support multiple eco-systems with a single implementation in their hardware platform.

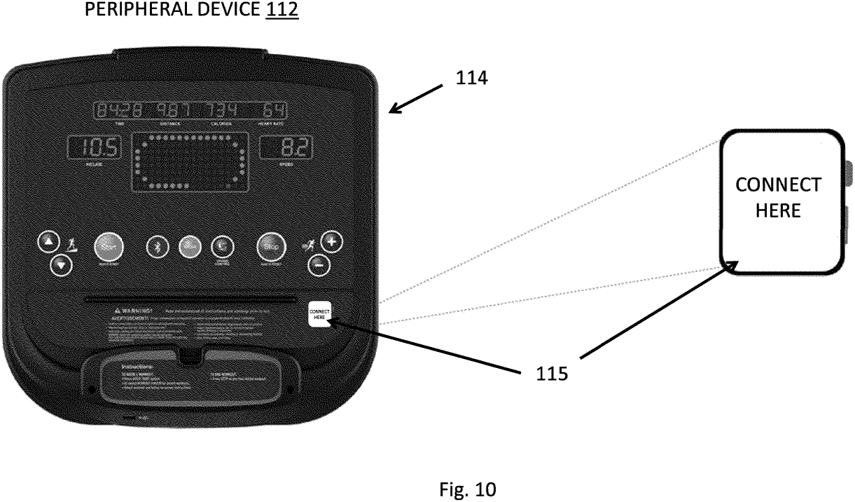

The console 114 can provide visual guidance on the fitness equipment 112 indicating where the mobile device 110 owner should place their mobile device 110 to establish the initial Bluetooth connection. For example, as shown in FIG. 10, a sticker, label, or other marker or indicator 115 can be placed near the location where the wireless connectivity module 122 is located on the console 114 indicating that the mobile device 110 owner should place their mobile device 110 at or near this location to connect to their mobile device 110 to the smart connectivity ecosystem application via console 114.

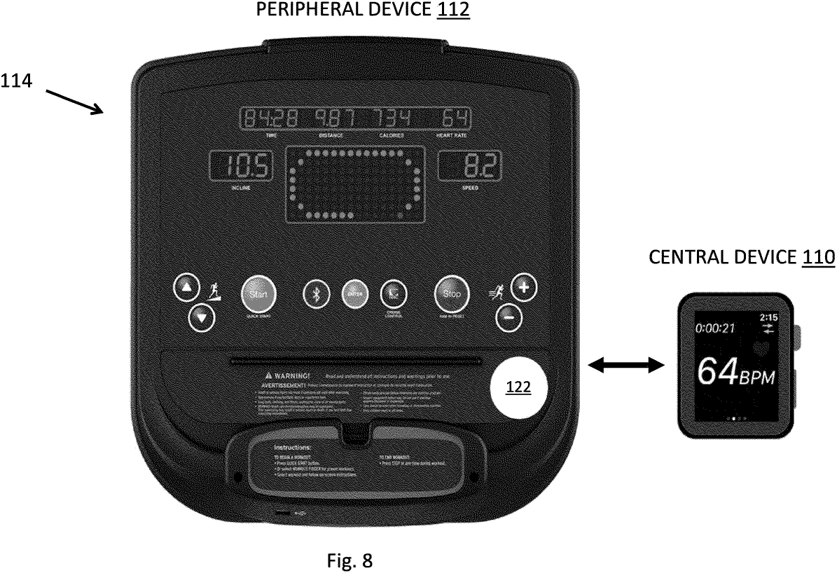

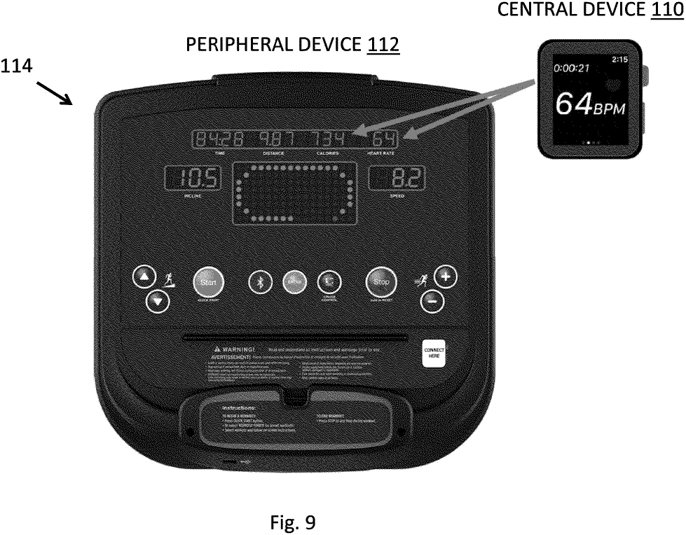

With reference now to FIGS. 8-10, additional use cases for various embodiments as described herein include connectivity technology such as a tap to pair methodology between a user’s mobile device 110 and various fitness equipment consoles 114 and the like. With reference to FIGS. 8 and 9, the BLE peripheral wireless connectivity module 122 can be utilized in a fitness equipment console 114 of a fitness machine 112.

FIG. 8 shows various components in a system and the roles each plays. As shown in FIG. 8, the console 114 of the fitness machine (peripheral device) 112 can initiate a (e.g., BLE) communication and service with the (central) mobile device 110 via a wireless connectivity module 122. FIG. 8 shows an operational flow for the smart connectivity ecosystem at a console 114 (e.g., including a module 122) and central mobile device 110. For example, heart rate and calories and the like can be transmitted from the mobile device 110 to the console 114. In some embodiments, the console 114 can provide workout metrics, messages, control signals, and the like to the user’s mobile device 110.

FIG. 9 shows various types of data that can be transferred between a mobile device 110 and a console 114. As shown in FIG. 10, the user can use the mobile device 110 to initiate a Bluetooth (e.g., BLE) radio connection to record the real-time metrics (see user’s heart rate of 64 BPM at [central] central device 110) on the console 114 by holding the mobile device 110 that is running the disclosed enabled application close to the pairing location on the console 114 to initiate a secure workout session with the machine’s console 114. A physical touch of the mobile device 110 to the console is preferably not required. Once initiated through proximity-based detection and pairing, a secure session between the properly enabled radio in the machine console 114 and the user’s mobile device 110 allows various data to flow bi-directionally between the machine console 114 and the mobile device 110 without any additional interaction needed from the user.

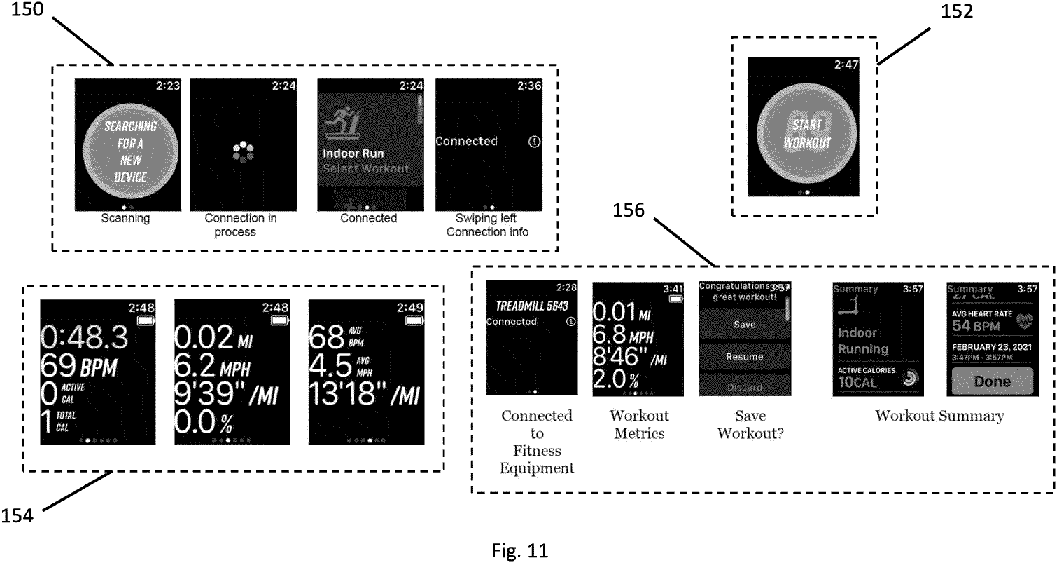

Additional views of the (central) mobile device 110 display during pairing and use are shown at FIG. 11. In more detail, FIG. 11 shows example graphical user interface and display screenshots of various embodiments of the present disclosure on a (central) mobile device 110. The shown examples utilize Applicant’s smart connectivity ecosystem for a smart watch mobile device 110. A first group of screenshots is shown at 150, and includes a scanning/searching view, a connection in process view, a connected view, and a connection info view. Shown at 152 is a display showing a “start workout” indicator for receiving a user input to start a workout. Shown at 154 are various views taken during an exercise session, including distance, heart rate, time, pace, calories, and the like. Shown at 156 are further display views as shown on mobile device 110, including a connected to fitness equipment view, a workout metrics view, a save workout? View, and various workout summary views.

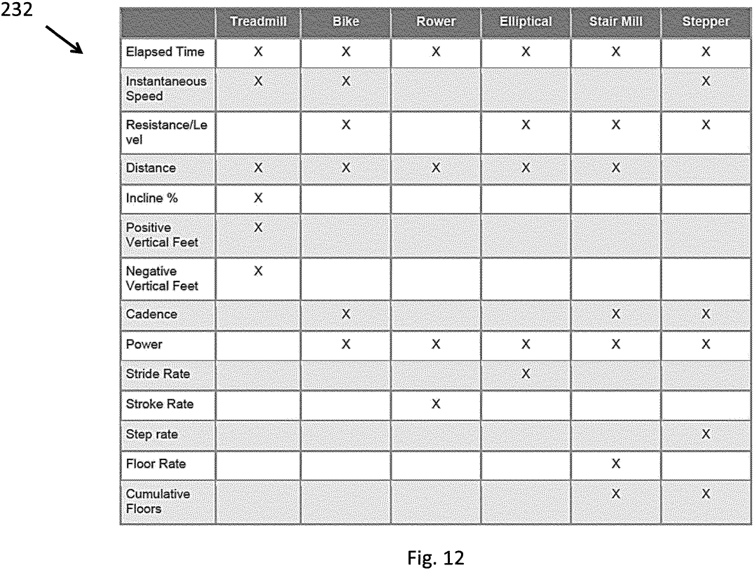

FIG. 12 shows example metrics communicated by or with a fitness equipment console 114, according to various embodiments.

FIG. 13 is a top view of an example network bridge 118 printed circuit board, including for example four radio modules (1-4), according to various embodiments.

Applicant further provides the following example details regarding a contemplated, optional, network bridge 118. As noted above, various embodiments herein utilize direct bi-directional communication between mobile device 110 and equipment 112 without the use of one or more network bridges 118. As applicable, various embodiments of the cloud 120 (e.g., using WASP SDK) and/or network bridges 118 can utilize power and/or data over Ethernet, also known as Power over Ethernet (PoE). Preferably, therefore, the various network bridges 118 are in a wired network connection with or without one or more central server (of cloud 120), although in other embodiments the network bridges 118 can communicate between one another using various wired or wireless standards, such as ANT, Bluetooth, Wi-Fi, etc. It is also contemplated that various network bridge 118 connection can utilize direct, TCP/IP connections for a non “broadcast” connection of a targeted mobile device, or the like in various embodiments. For instance, there may be situations where an individual user may desire a mobile device 110 or fitness equipment 112 connection that is passed directly as opposed to a broadcast. A TCP/IP connection protocol can be beneficial in an environment or facility where the layout of the radio frequency (RF) environment does not allow for the various network bridge 118 nodes to communicate with each other using standard RF transport layers. Additionally, a TCP/IP protocol allows for additional options for interfacing with existing networking infrastructure.

Specifically, Applicant’s WASP-PoE, PoE device is an example of the network bridge 118 described herein. The WASP-PoE, also referred to herein as a PoE network bridge 118, is a bridge for BLE and/or ANT+ devices to communicate through a wired Ethernet network to any designed device connected to the same network. Powered either by the voltage on the Ethernet cable (PoE support) or auxiliary 36-72V DC input. Integrating an 8-channel BLE/ANT+ receiver with Ethernet circuitry, the PoE network bridge provides a data gateway for monitoring, recording and analyzing BLE/ANT+ data in designated network locations with lower overhead than the standalone network bridge. The PoE network bridge 118 receives data from connected BLE/ANT+ devices and forwards the data through the Ethernet network to the end points on the Ethernet network. The PoE network bridge 118 Application Programming Interface (API) can be exactly the same as for other network bridges (e.g., WASP), which is an open API that is used by developers to integrate the PoE network bridge 118 into BLE/ANT+ sensor monitoring and control applications. To use the PoE network bridge 118 in an Ethernet network, preferably the network meets certain specifications.

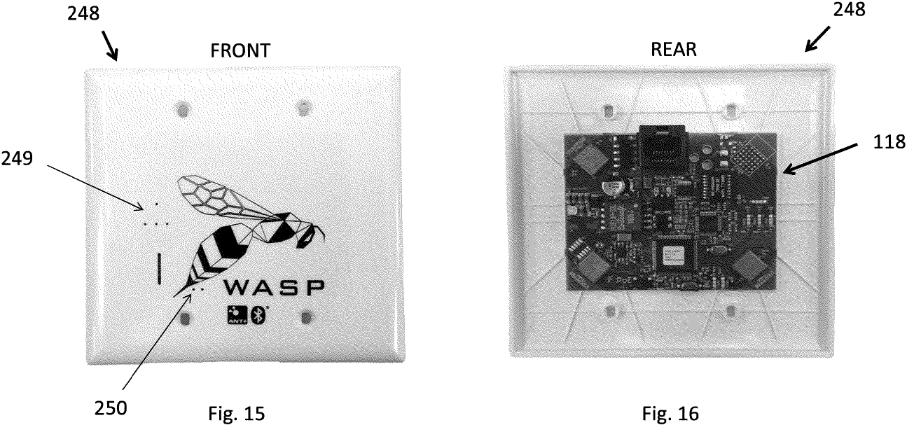

Example PoE network bridge 118 specifications can include various ethernet protocols, including 10/100 Ethernet, power source can be line powered PoE or PoE injector at 36-72 VDC, can utilize Modes A and B for PoE power, and can use less than 1 W power consumption. In various embodiments, 1-4 radio configurations are contemplated, and an ANT+ radio frequency based is for example 2.4 GHz ISD (2.40000-2.4835 GHz). An example ANT+ radio data rate is 1 Mbps, and an example ANT+ radio modulation is GFSK. See also FIG. 15.

FIG. 14 is a top view of an example (GEM2/GEM3) wireless connectivity module 122, provided on a printed circuit board, according to various embodiments. As shown, the module 112 includes mounting holes 236, a USB (or other) connector 237. The USB connector 237 can be used to provide power to the wireless connectivity module 122. The USB connector is also optionally used to connect the wireless connectivity module 122 to a computer running a console simulator (e.g., a GEMHCI Console Simulator, such as for testing and configuration). The wireless connectivity module 122 also includes one or more pin headers 238, which can be used to connect the module 122 to an external prototype circuit board for prototyping, etc. Also optionally included are various LEDS, such as various link and/or green power LEDs, which can indicate when the wireless connectivity module 122 is properly powered.

Applicant produces various versions of the shown communication module 122. Some versions of the communication module 122 include firmware to support Applicant’s various Bluetooth services for data exchange between the central mobile device 110 and the wireless connectivity module 122. Through applications on the mobile device 110, the module 122 receives heart rate and calorie data from the mobile device 110. The module 122 then passes any data it receives such as workout metrics from a fitness equipment console 114 over the same Bluetooth connection with the mobile device 110 for logging into an applicable, e.g., mobile device, application.

As discussed herein, the wireless module 122 shown in FIG. 14 can be utilized in various cases, including a (WASP) network bridge 118, or fitness equipment 112 itself. Applicant’s specific (WASP3) network bridge 118 application with the wireless module 122 can be somewhat different than the fitness equipment 112 application of the wireless module 122. The wireless module 122 (version 3) in this case implements the Applicant’s Bluetooth service for data exchange between the mobile device 110 and the network bridge 118. The mobile device 110 would still pass the same data (e.g., heart rate and calories) but the network bridge 118 may pass other data from a networked application, e.g., one that implements Applicant’s proprietary WASP SDK. The other data could include a user’s workout metrics, but also could include other data or functions such as workout start/stop/pause commands to start/stop/pause the workout in the mobile device 110 or equipment 112 application. Other data received from the network bridge(s) 118 could also include short messages, including text, photos, sound, etc. such as “your fitness appointment is upcoming soon,” “your smoothie is ready,” or “child care needs you,” for example.

As discussed herein, various examples of the wireless connectivity module 122 contemplated can utilize features of Applicant’s various wireless (GEM) connectivity modules. The wireless connectivity modules as described below are therefore examples of the wireless connectivity module 122 used herein.

The following are example details of one example wireless connectivity module (version 2, GEM2), and any details as follows can be incorporated into any embodiments herein. The example wireless connectivity module has been designed to allow OEMs to easily add Bluetooth, ANT+ connectivity in their product offering. The wireless connectivity module incorporates Applicant’s console and simulation protocol software specifically designed to enable fitness machines such as treadmills, exercise bikes, ellipticals, stair climbers and step machines to wirelessly communicate exercise data with the smart watch, smart phones, tablets, other fitness watches, and leaderboard software systems using standard Bluetooth and ANT+ services and profiles.

The wireless connectivity module’s onboard software supports Bluetooth FTMS, Bike Power, Cycling Speed and Cadence, legacy GymConnect, and Applicant’s smart connectivity ecosystem application and ecosystem. The wireless connectivity module also supports simultaneous ANT+ communications using FE-C, Bike Power, and Cycling Speed and services. By default, the wireless connectivity module automatically enables Bluetooth FTMS and ANT+ FE-C. Bike Power and cycling Speed cadence service/profile support can be enabled through the various protocols, such as the GEMHCI protocol.

The wireless connectivity module is based on Nordic Semiconductor’s nRF52832 multi-protocol Bluetooth and ANT+ chipset. The wireless connectivity module offers a UART host interface and has a maximum transmit power of +4 dBm, and a sensitivity of -96 dBm. This guide is intended to assist the engineer in becoming familiar with the wireless connectivity module’s smart connectivity ecosystem mobile device connectivity feature using a wireless connectivity module development board.

FIGS. 15 and 16 show an example of an example network bridge 118 on an installable wall plate 248 with various ethernet LED indicators 249 and status LEDs 250.

FIG. 15 shows (e.g., PoE network bridge) indicator LED 249 locations, and the function of each LED 249 as shown is for example: top: G4; bottom (from left to right): G3, G2, G1. The Ethernet indicator LEDs 249 are optionally green, and an example operation is defined below. G1 = Ethernet Link Good. G2 = Ethernet Link Speed (ON=100 Mb/s, OFF=10 Mb/s). G3 = Ethernet Link Activity. G4 = Power Indicator. Examples of the PoE network bridge can be made to fit into a standard dual-gang electrical box. FIG. 16 shows an example network bridge 118 on the installable wall plate 248 from a rear view. With reference again to FIG. 13, example (PoE radio) network bridges 118 can be equipped with for example 1 to 4 radios (e.g., wireless connectivity modules 122) as shown as radios 1-4. More than 4 radios can optionally be located on network bridge 118.

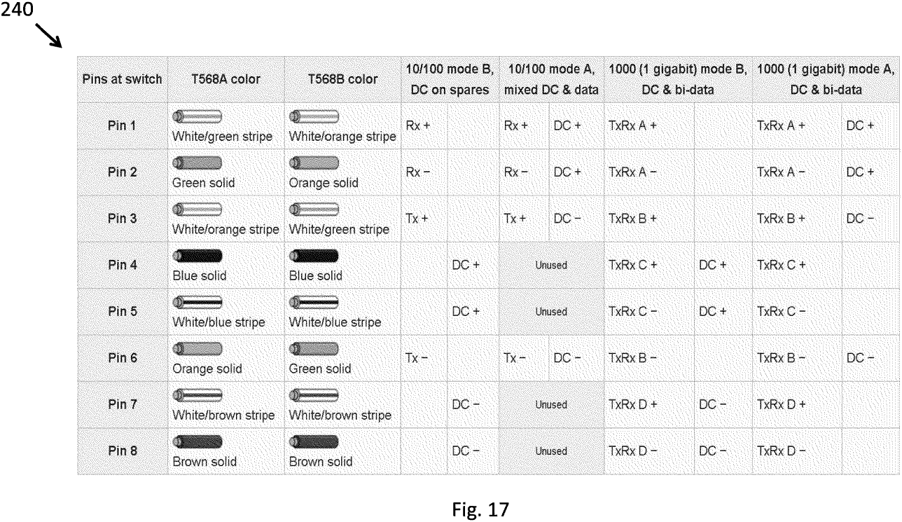

If a (e.g., fitness) facility has a powered Ethernet network, then the (optionally PoE) network bridge 118 can be plugged into the network and it can be powered thereby. The network bridge 118 may contact the applicable server, requesting a DHCP address. If the network addressing is not managed by a DHCP server, the network bridge 118 may fail to connect to the network. In various embodiments, the network bridge 118 wiring supports both Modes A & B for PoE power. See example network bridge 118 pinout chart 240 of FIG. 17 for additional formation. If a powered Ethernet network is not present or available, then a PoE “Power Injector” can be used to power the network bridge 118. Preferably, an input voltage range from the power injector to the network bridge 118 should be in the 36-72V range. If multiple network bridges 118 are being utilized, a multi-port power switch or multiple power injectors, one for each network bridge 118, can be used. A single port PoE injector can be used with the network bridge 118, e.g., the Intellinet 1-Port Power over Ethernet Injector.

FIG. 17 shows an example wiring pinout chart 240 for an example (e.g., WASP) optional network bridge 118, according to various embodiments. As shown, the pinouts of chart 240 include 802.3af Standards A and B from power sourcing equipment perspective.

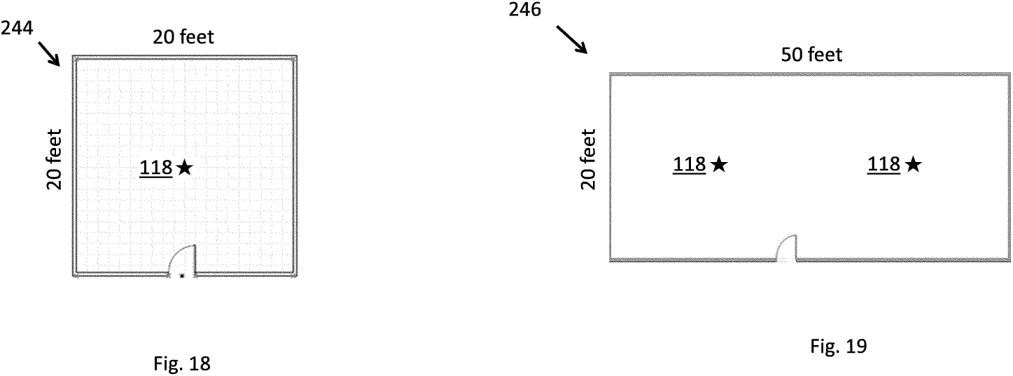

FIG. 18 shows an example preferred position for a network bridge 118 in a room 244 of a first size, according to various optional embodiments. FIG. 19 shows an example preferred position for two network bridges 118 in a room 246 of a second size, according to various optional embodiments. An example of the first size room is 20 feet by 20 feet, and an example of the second room size is 20 feet by 50 feet.

If one or more network bridge 118 is utilized and placed within a space or room, in general a PoE network bridge 118 will operate satisfactorily anywhere within a room of up to approximately 400 square feet. This limitation is due to example radios (e.g., BLE, ANT+) in sensor devices. Sensor devices use a very low power radio, so the signal doesn’t travel very far. For a PoE network bridge 118 to cover an average room of approximately 20-by-20 feet, it should be placed in the center of the room as shown in FIG. 18. Very large rooms may have difficulty receiving ANT+ data from devices furthest from the PoE network bridge 118. This can be easily remedied by adding additional PoE network bridges 118 in the room. Suggested placement for a larger wide room is shown in FIG. 19. Preferably, two PoE network bridges 118 are placed equidistant between the front and back walls. Then divide the length of the room by 4 and place each PoE network bridge 118 that distance from the side walls. This effectively splits the room into two halves, with each PoE network bridge 118 covering one half of the room. Ideally, the PoE network bridge 118 is placed as high as possible at the indicated position in the diagrams of FIGS. 18 and 19. This provides the best unobstructed path for signals from the wireless-equipped devices to reach each PoE network bridge 118. Some devices may be “heard” by both PoE network bridge 118 units. If an application uses either the WASP Class Library or Framework, an application can recognize this condition and de-duplicate the messages. In other embodiments, no network bridges 118 are utilized.

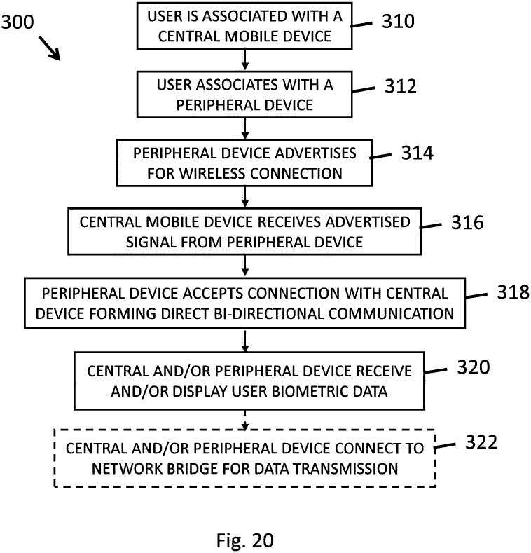

FIG. 20 shows a fitness environment connectivity and network scheme process 300, according to various embodiments. The process can start at 310 with a user being associated with a central mobile device (e.g., 110 as used herein). The user can then associate with a peripheral device (e.g., 112 as used herein) at 312. At 314, the peripheral device can then advertise for wireless connection (e.g., BLE). At 316, the central mobile device receives the advertised signal from the peripheral device. At 318, the peripheral accepts the connection with the central device (e.g., by a handshake) and bi-directional wireless communication (e.g., via BLE) is formed. At 320, the central and/or peripheral device receives and/or displays user biometric/fitness/sensor data. At optional step 322, the central and/or peripheral device connects to one or more network bridge (e.g., 118 as used herein) for data transmission. As discussed herein, the network bridge, if present, can be connected to various servers and/or cloud 120 environments.

According to various embodiments and to protect user privacy, any data sent across the BLE (or other wireless) connection between any of the user mobile device 110, fitness machine 112, wireless network bridges 118, and data cloud 120 can be encrypted end to end. This encryption secures any data being transmitted, preventing so-called man-in-the-middle attacks or external BLE sniffers from collecting sensitive user information. Also related to security aspects, some mobile devices, such as Apple Watch, may only operate as a central device, e.g., for control of advertisement to various other potential devices for connection.

As mobile devices 110, such as smart watches and mobile phones, add more sensing capabilities the need to allow access to the sensor data will continue to grow. The described connectivity model allows device owners the ability to share this sensor data with any system capable of communicating using this protocol methodology. Other examples, which may or may not be wellness or fitness related, include monitoring the stress level of employees, especially in high-stress environments, monitoring users' personal body temperature to identify either personal safely, or indicate a potential viral or other communicable infection, such as COVID-19. Other use cases extend well beyond the examples included herein, and there are nearly limitless use cases for user data and other data sharing across diverse platforms for diverse uses.

A specific embodiment is described in greater detail, below.

### Embodiment 1

According to this embodiment, a system utilizes an Apple Watch as the mobile device 110, GEM module(s) as the wireless connectivity module(s) 122, WASP data transceiver(s) as optional network bridge(s) 118, and Applicant’s Heartbeatz as the example smart connectivity ecosystem.

The Heartbeatz smart connectivity ecosystem application utilizes the Apple Watch OS HealthKit framework to access the heart rate and calorie data measured by the Apple Watch. To share the heart rate and calorie data measured by the Apple Watch, the smart connectivity ecosystem application will scan for a smart connectivity ecosystem capable receiver advertising Applicant’s custom smart connectivity ecosystem Service. The heart rate and calorie data are shared once the smart connectivity ecosystem application and the smart connectivity ecosystem receiver device establish a Bluetooth connection with each other. Workout metrics from the GEM2 enabled equipment are logged in real time and can be saved to the Apple Watch owner’s activity history on the Apple Watch, giving the Apple Watch owner move ring, and workout ring credit.

Compatible GEM Firmware Version: The smart connectivity ecosystem Apple Watch application support is included in GEMHCI firmware versions 1.11.3 and newer.

GEM Module Configuration and Events: To take advantage of the smart connectivity ecosystem Apple Watch application and GEM module integration, the following section outlines the GEM settings and GEMHCI protocol events are preferably enabled/accommodated by the fitness equipment console.

Fitness equipment type: The smart connectivity ecosystem application uses the equipment type configured by the console to automatically set the workout type recorded by the app. It is important then for the console to set the equipment type using the GEMHCI command SET FITNESS EQUIPMENT TYPE. Examples of equipment types currently recognized by the smart connectivity ecosystem application include: treadmills, indoor (stationary) bikes, ellipticals, stair/steppers, and rowers.

Preferably a Bluetooth name should be configured to include the equipment type and a random 4-digit number that changes after each workout session appended at the end. The Bluetooth workout session name should be displayed on the console when Bluetooth is initiated by the equipment user. Using the equipment type name and random number and displaying the unique workout session name makes it easier for equipment users to the correct fitness equipment to log their workout. The Bluetooth Name is set using the GEMHCI SET DEVICE NAME command.

With respect to Bluetooth advertising, the GEM module with smart connectivity ecosystem support can support up to 3 simultaneous Bluetooth connections. Supporting 3 simultaneous Bluetooth connections would allow, for example, the GEM module connections with the Apple Watch smart connectivity ecosystem app, Zwift or similar workout app, or another Apple Watch when doing team workouts on the same equipment. To establish a Bluetooth connection, the console will need to enable the GEM module’s Bluetooth radio by using the GEMHCI START ADVERTISING command. An application such as the smart connectivity ecosystem Apple Watch application that is scanning for the GEM module, can then connect to the GEM module once advertising has been initiated. After each connection is established, the console will need to trigger the GEM module to advertise again using the GEMHCI START ADVERTISING command and appropriate console user interface (UI). The Bluetooth name will be the same during each workout session for each connection. Once the three connections are made, if the user attempts to connect a 4th device, the GEM module will send an error event to the console indicating the maximum number of Bluetooth connections has been reached.

Regarding equipment operation messages, and with the two-way Bluetooth link between the smart connectivity ecosystem Apple Watch application and the GEM2 module, the fitness equipment console with the GEM2 module will send equipment state operations to the smart connectivity ecosystem application to start/pause/end the workout tracking feature of the smart connectivity ecosystem app. Equipment states are communicated to the GEM module by the console using the GEMHCI command SET FITNESS EQUIPMENT STATE.

For real-time workout metric logging, when the smart connectivity ecosystem application is connected, the GEM module with smart connectivity ecosystem firmware will automatically send workout metrics sent to the GEM module by the console using the GEMHCI command UPDATE WORKOUT DATA. In various embodiments, consoles 114s can send any metrics as outlined in the non-exhaustive table 232 of FIG. 12.

For heart rate and calorie data events, the smart connectivity ecosystem Application will send real-time heart rate and calories measured by the Apple Watch to the GEM module over the Bluetooth connection. The GEM module will send the real-time heart rate and calorie data from the Apple Watch to the console using the GEMHCI Events HEART RATE DATA RECEIVED EVENT and CALORIE DATA RECEIVED EVENT. It is recommended that the fitness equipment console display the calorie data received from the smart connectivity ecosystem application in lieu of the calorie data calculated by the console to eliminate confusion that could occur when the calories calculated by the fitness console and the Apple Watch differ. As shown in FIG. 10, it is further recommended that the fitness equipment console display the real time heart rate data measured and sent by the Apple Watch where it would normally display HR data received from a chest strap or hand heart rate sensors.

1. A system, comprising:
a mobile device associated with a user; and
a smart fitness equipment unit,
wherein the mobile device is a central device and the smart fitness equipment unit is a peripheral device in communication with the mobile device.

2. The system of claim 1, wherein at least the mobile device and the smart fitness exercise equipment communication using Bluetooth Low Energy (BLE).

3. The system of claim 1, wherein the mobile device is a wearable device.

4. The system of claim 3, wherein the wearable device is a smart watch.

5. The system of claim 1, wherein the smart fitness equipment unit advertises a connection, and the mobile device responds to initiate a paired connection.

6. The system of claim 1, wherein the mobile device is configured to make requests of at least the smart fitness equipment unit.

7. The system of claim 1, wherein the smart fitness equipment unit comprises at least one wireless connectivity module.

8. The system of claim 7, wherein the at least one wireless connectivity module of the smart fitness equipment unit is provided in a console.

9. The system of claim 8, wherein a connection between the mobile device and the console of the smart fitness equipment is initiated when the user positions the mobile device proximate a wireless connectivity module of the console.

10. The system of claim 1, wherein after the mobile device is in communication with the smart fitness equipment unit, the mobile device and the smart fitness equipment unit can communicate directly with each other.

11. A method for providing connectivity in a fitness center environment, comprising:
providing a plurality of mobile devices;
associating the plurality of mobile devices with a corresponding plurality of users;
providing a plurality of smart fitness equipment units; and
creating a network connecting at least one of the plurality of mobile devices and at least one of the plurality of fitness equipment units;
wherein each mobile device of the plurality of mobile devices is a central device and each smart fitness equipment unit is a peripheral device in direct communication with at least one central device of the plurality of mobile devices.

12. The method of claim 11, further comprising a reception area, wherein the system utilizes a user’s mobile device to identify the user’s identity using at least a wireless connectivity module.

13. The method of claim 11, further comprising a locker room comprising a plurality of lockers each comprising a wireless connectivity module, wherein at least a first locker of the plurality of lockers is assigned to a user, and is caused to be unlocked when the user’s mobile device approaches the wireless connectivity module of the first locker and optionally when the user further indicates the first locker is to open.

14. The method of claim 11, wherein the user can seamlessly transition from one smart fitness equipment unit or fitness class or room to another fitness equipment unit or fitness class or room without direct user intervention.

15. The method of claim 11, wherein each of the plurality of mobile devices and each of the plurality of smart fitness equipment units are provided with a wireless connectivity module.

16. A method of connecting a user mobile device to a smart fitness equipment unit, comprising:
broadcasting an availability signal from a peripheral smart fitness equipment unit;
listening for an availability signal using a central mobile device;
initiating a handshake operation between the peripheral smart fitness equipment unit and the central mobile device; and
establishing a direct bi-directional connection between the peripheral smart fitness equipment unit and the central mobile device.

17. The method of claim 16, wherein the central mobile device is a wearable device.

18. The method of claim 16, wherein the availability signal and the direct bi-directional connection utilize Bluetooth Low Energy (BLE).

19. The method of claim 16, wherein each of the between the peripheral smart fitness equipment unit and the central mobile device are equipped with a wireless connectivity module.

20. The method of claim 16, wherein the establishing the direct bi-directional connection between the mobile device and console of the smart fitness equipment is initiated when the user positions the mobile device proximate a console of the peripheral smart fitness equipment unit.
